(19) United States
(12) Patent Application Publication
(10) Pub. No.: US 2020/0273554 A1
(43) Pub. Date: Aug. 27, 2020

(54) Systems and Methods for Medication Intake Monitoring

(71) Applicant: BI Incorporated, Boulder, CO (US)

(72) Inventors: Duke Hanson, Boulder, CO (US); Dustin Pettit, Boulder, CO (US)

(73) Assignee: BI Incorporated, Boulder, CO (US)

(21) Appl. No.: 16/599,395

(22) Filed: Oct. 11, 2019

Related U.S. Application Data
(60) Provisional application No. 62/810,217, filed on Feb. 25, 2019.

(51) Int. Cl. G16H 20/10; G16H 10/60; G16H 40/67; H04N 7/18

(52) U.S. Cl. CPC G16H 20/10; H04N 7/183; G16H 40/67; G16H 10/60

(57) ABSTRACT

Various embodiments provide systems and methods for monitoring medication intake by a monitored individual.

### CROSS REFERENCE TO RELATED APPLICATION

The present application claims priority to (i.e., is a non-provisional of) U.S. Pat. App. No. 62/810,217 entitled “Automated Medication Tracking for Clients”, and filed Feb. 25, 2019 by Pettit et al. The entirety of the aforementioned application is incorporated herein by reference for all purposes.

### BACKGROUND OF THE INVENTION

Various embodiments provide systems and methods for monitoring medication intake by a monitored individual.

Large numbers of individuals are currently monitored as part of parole or other requirements. Such monitoring allows a monitoring agency to determine whether the individual is engaging in acceptable patterns of behavior, and where an unacceptable behavior is identified to work to discourage such behavior going forward. In many cases individuals subject to monitoring are required to take one or more medications. As the failure to take such medications may impact behavioral tendencies, it is important to have some insight into whether the individual is taking the medications. Often the taking of medications is monitored through questioning of the individual which can be unreliable, or by having the individual visit a monitoring authority to take the medication in their presence which can be both intrusive and costly.

Thus, for at least the aforementioned reasons, there exists a need in the art for more advanced approaches, devices and systems for monitoring.

### BRIEF SUMMARY OF THE INVENTION

Various embodiments provide systems and methods for monitoring medication intake by a monitored individual.

This summary provides only a general outline of some embodiments. Many other objects, features, advantages and other embodiments will become more fully apparent from the following detailed description, the appended claims and the accompanying drawings and figures.

### BRIEF DESCRIPTION OF THE DRAWINGS

A further understanding of the various embodiments may be realized by reference to the figures which are described in remaining portions of the specification. In the figures, similar reference numerals are used throughout several drawings to refer to similar components. In some instances, a sub-label consisting of a lower case letter is associated with a reference numeral to denote one of multiple similar components. When reference is made to a reference numeral without specification to an existing sub-label, it is intended to refer to all such multiple similar components.

FIG. 1a is a block diagram illustrating a medication intake monitoring system including a user detached monitor device in accordance with various embodiments;

FIG. 1b is a block diagram of a user detached monitor device usable in accordance with one or more embodiments;

FIGS. 2a-2c are flow diagrams showing a method in accordance with some embodiments for monitoring medication intake by a monitored individual;

FIGS. 3a-3f are graphical depictions of the processes set forth in the flow diagrams of FIGS. 2a-2c;

FIG. 4a is a block diagram illustrating a hybrid medication intake monitoring system including a user detached monitor device and a user attached monitor device in accordance with various embodiments;

FIG. 4b is a block diagram of a user detached monitor device usable in accordance with one or more embodiments;

FIG. 4c is a block diagram of a user attached monitor device usable in accordance with one or more embodiments;

FIG. 4d shows a user attached monitor device with an attachment element for attaching the user attached monitor device to a limb of an individual in accordance with some embodiments; and

FIG. 5 is a flow diagram showing a method in accordance with some embodiments for maintaining a count of remaining doses of a medication and to automatically order refills of the medication.

### DETAILED DESCRIPTION OF THE INVENTION

Various embodiments provide systems and methods for monitoring medication intake by a monitored individual.

It has been found that returning offenders to society after being locked up in a secure facility with little if any control of their day to day activities is often unsuccessful. It is often helpful to have, for example, a parole officer or other monitoring official monitor their movements and activities for a period of time as they reenter society. In some cases, the parole officer is aided by a tracking device attached to the individual being monitored. However, this is costly as a parole officer must be significantly involved in monitoring and responding to situations. Some embodiments disclosed herein reduce the interaction between the tracking device and the parole officer.

Further, it has been found that some offenders are much more likely to engage in problematic behavior when they fail to take medications prescribed to them. In some cases, the monitored individuals are required to take the prescribed medications as part of the terms of their parole, release, or probation. In such cases, it is beneficial for the monitoring officer, parent, or other supporting individual to have some ability to discern whether the monitored individual is taking their medications. Some embodiments disclosed herein allow for remote monitoring of medication intake.

Some embodiments provide systems for monitoring client medication. Such systems include a client device having: a camera, an electronic interface operable to communicate instructions to the client, a wireless transceiver operable to transfer data from the client device to a remote monitoring receiver, and a computer readable medium including non-transient instructions. The non-transient instructions are executable by a processor to: communicate a first instruction via the electronic interface requesting that the client identify a medication to be taken, determine that the client has properly identified the medication to be taken, communicate a second instruction via the electronic interface requesting that the client position the client device to capture at least one image, receive the at least one image from the camera; determine that the image complies with the instruction, and based upon the determination that the image complies with the instruction, transmit the at least one image via the wireless transceiver to a remote monitor device.

In some instances of the aforementioned embodiments, the client device is a user detached monitor device. In other instances, the client device is a combination of a user detached monitor device and a user attached monitor device.

In various instances of the aforementioned embodiments, the computer readable medium further includes instructions executable by the processor to: receive an indication of the medication via the wireless transceiver, receive a schedule indicating when the medication is to be taken. In such instances, the first instruction is communicated via the electronic interface at a time identified in the schedule. In some instances, the first instruction requests that the client position the client device such that the camera can take an image of the medication, and the computer readable medium further includes instructions executable by the processor to transmit the image of the medication via the wireless transceiver to the remote monitor device. In one or more instances the first instruction requests that the client position the client device such that the camera can take an image of the medication, and the computer readable medium further includes instructions executable by the processor to transmit the image of the medication via the wireless transceiver to the remote monitor device. In other instances of the aforementioned embodiments, the first instruction requests that the client position the client device such that the camera can take an image of the medication, and the computer readable medium further includes instructions executable by the processor to automatically determine that the image of the medication is the medication to be taken.

In some instances of the aforementioned embodiments, the second instruction requests that the client position the client device to capture a first image of the client's open mouth with the medication to be taken placed therein, and to subsequently capture a second image of the client's open mouth after the client has swallowed the medication to be taken. In one or more instances of the aforementioned embodiments, the second instruction requests that the client position the client device to capture a first image of the client's open mouth with the medication to be taken placed therein, and to subsequently capture a second image of the client's open mouth after the client has swallowed the medication to be taken, wherein the second image shows a top side of the client's tongue. In some instances of the aforementioned embodiments, the second instruction requests that the client position the client device to capture a first image of the client's open mouth with the medication to be taken placed therein, and to subsequently capture a second image of the client's open mouth after the client has swallowed the medication to be taken, and the second image shows a bottom side of the client's tongue.

In some instances of the aforementioned embodiments, the electronic interface includes an audio interface. In various instances of the aforementioned embodiments, the electronic interface includes a visual display interface. In one or more instances of the aforementioned embodiments, determining that the image complies with the instruction includes comparing the at least one image with a previously stored image. In some instances of the aforementioned embodiments, determining that the client has properly identified the medication to be taken includes determining that the client has responded to the first instruction within a preset time period. In various instances of the aforementioned embodiments, determining that the image complies with the instruction includes determining that the client has responded to the second instruction within a preset time period.

Other embodiments provide methods for monitoring client medication. The methods include: sending a message to a monitored individual via a client device prompting the monitored individual to take a medication; receiving a first image from a client device showing a container of the medication; prompting the individual to perform a multi-step process to take the medication; receiving a second image from the client device showing the medication inside the mouth of a monitored individual associated with the client device; receiving a third image from the client device showing the mouth of the monitored individual with a tongue of the individual in a down position; receiving a fourth image from the client device showing the mouth of the monitored individual with a tongue of the individual in an up position; and based upon a combination of the first image, the second image, the third image, and the fourth image, determining that the monitored individual successfully completed taking the medication.

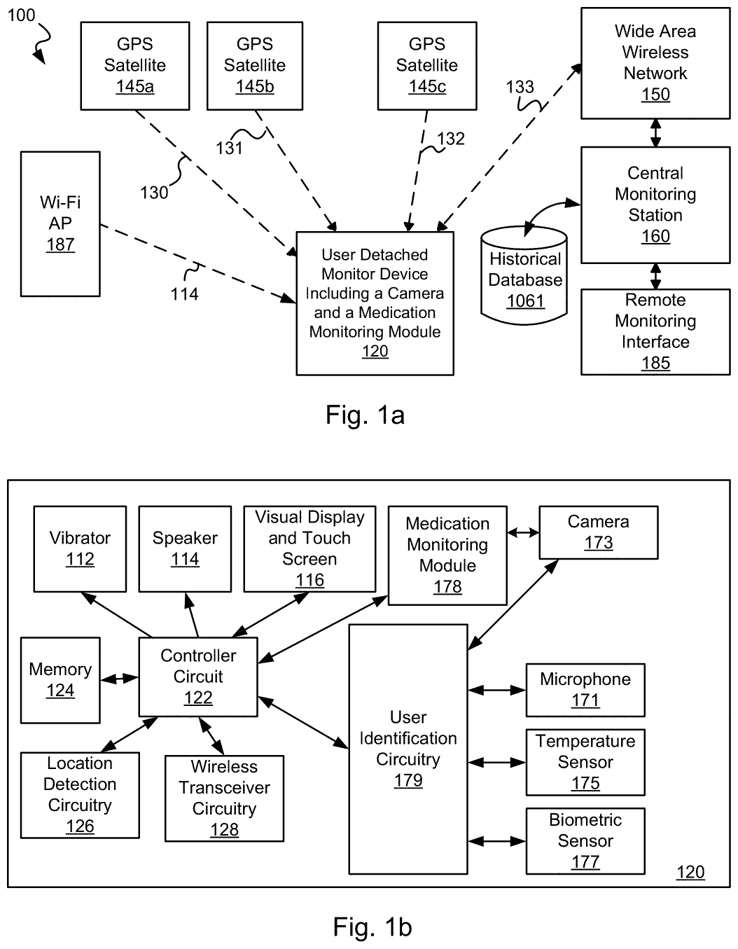

Turning to FIG. 1a, a block diagram illustrates a medication intake monitoring system 100 including a user detached monitor device 120 acting as a client device in accordance with various embodiments. User detached monitor device 120 includes a medication monitoring module operable to enable monitoring of a medication by an individual associated with user detached monitor device 120. User detached monitor device 120 is portable, and may be any device that is recognized as being used by or assigned to an individual being monitored, but is not physically attached to the individual being monitored by a tamper evident attaching device. User detached monitor device 120 may be, but is not limited to, a mobile telephone. A central monitoring station 160 is wirelessly coupled to user detached monitor device 120 via one or more wide area wireless (e.g., cellular telephone network, Internet via a Wi-Fi access point, or the like) communication networks 150 and a wireless communication link 133.

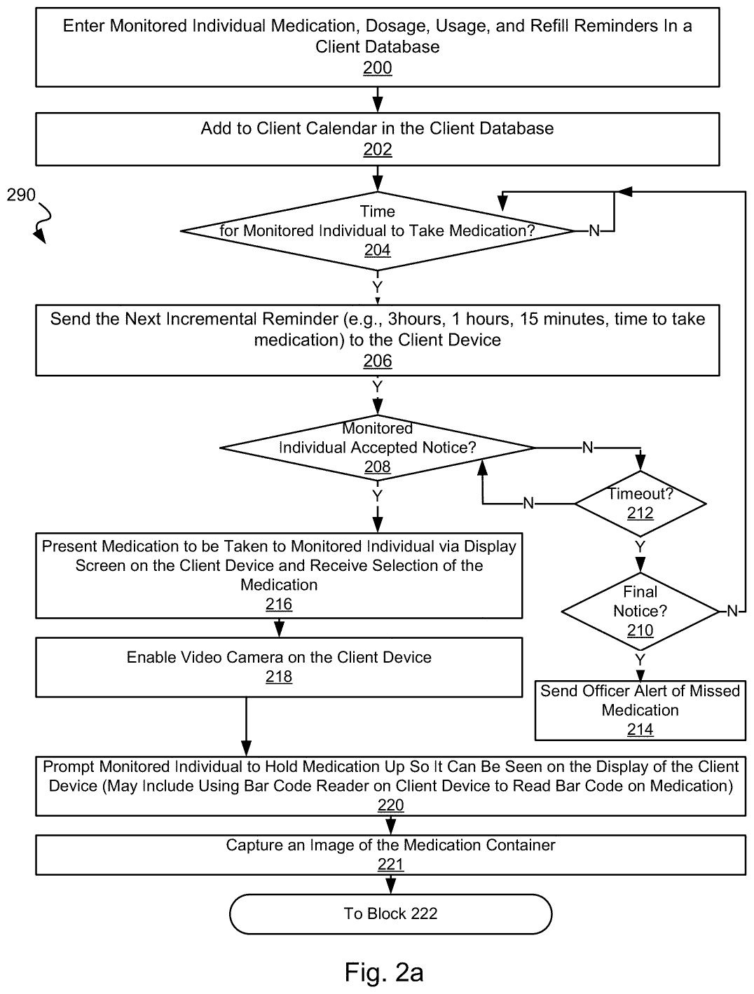

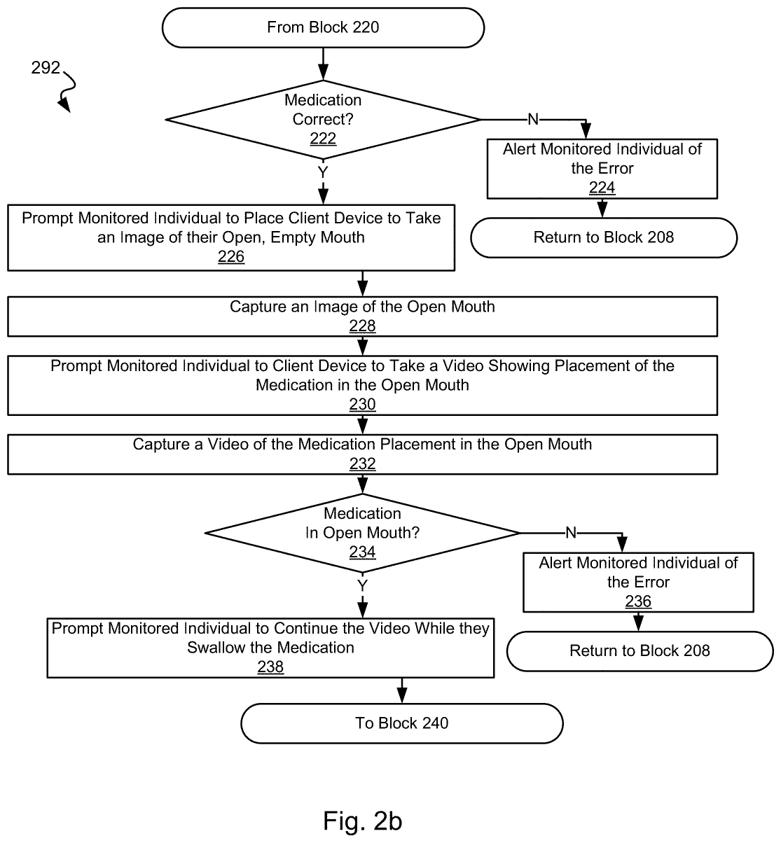

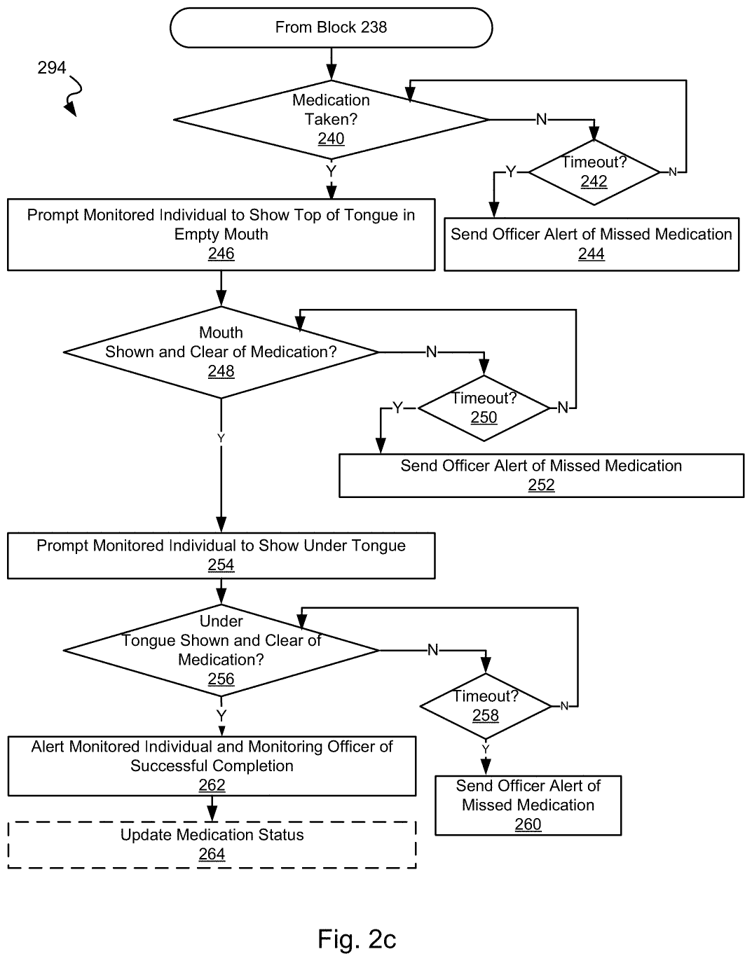

User detached monitor device 120 includes a medication monitoring module that operates to request and process information corresponding to the taking of medication by the individual associated with user detached monitor device 120. In some embodiments, the medication monitoring module causes a message to be displayed to the individual associated with user detached monitor device 120 indicating a medication that is to be taken. The medication monitoring module waits for a response from the monitored individual that they are ready to take the medication. The camera of user detached monitor device 120 is used to record the type of medication being taken followed by recording an image of the medication in the monitored individual's mouth, followed by subsequent image of the individual's mouth with the tongue down after the medication is swallowed, and then followed by a subsequent image of the individuals mouth with the tongue up after the medication is swallowed. All of these images can be shared with a monitoring officer either in real time or stored for later review. In some embodiments, the medication monitoring module includes image processing software operable to automatically discern the medication placed in the monitored individual's mouth from the image taken of the mouth, and then later discern that the medication is no longer in the monitored individual's mouth based upon the subsequent two images with the tongue up and the tongue down, respectively. FIGS. 2a-2c discussed in detail below show one embodiment of processes performed by a medication monitoring module.

The aforementioned medication intake monitoring data and/or other data gathered by user detached monitor device 120 is wirelessly transmitted to central monitoring station 160 via wide area wireless network 150. Central monitoring station 160 may be any location, device or system where the location data is received, including by way of non-limiting example: a cellular/smart phone, an email account, a website, a network database, and a memory device. The location data is stored by central monitoring station 160 and is retrievable therefrom by a monitor, such as a parent, guardian, parole officer, court liaison, spouse, friend, or other authorized group or individual. In this manner, the monitor is able to respond appropriately to the detected out-of-bounds activity by a monitored individual. In some cases, the monitor is able to retrieve the location data via a remote monitoring interface 185 which may be, but is not limited to, a network connected user interface device communicatively coupled via a network to central monitoring station 160 and/or directly to user detached monitor device 120 via wide area wireless network 150.

Additionally, user detached monitor device 120 may include a location detection circuit that senses the location of the client device and generates a location data. The location data may comprise one or more of: global positioning system (“GPS”) data, assisted GPS (“A-GPS”) data, advanced forward link trilateration (“AFLT”) data, and/or cell tower triangulation data. Where GPS is used, user detached monitor device 120 receives location information from three or more GPS satellites 145a, 145b, 145c via respective communication links 130, 131, 132. The aforementioned location data is utilized to verify the location of a user associated with user detached monitor device 120 at various points as more fully discussed below. User detached monitor device 120 is considered “ambiguous” because it is not attached to the user in a tamper resistant/evident way, but rather is freely severable from the user and thus could be used by persons other than the target. User detached monitor device 120 may additionally include a Wi-Fi transceiver capable of receiving information from one or more Wi-Fi access points 187 that can be used to discern location via a Wi-Fi communication link 114. As mentioned above, the location data gathered by user detached monitor device 120 may be wirelessly transmitted to central monitoring station 160 via wide area wireless network 150 accessed via a wireless link 133.

User detached monitor device 120 may further include a user identification sensor operable to generate user identification data for identifying the user in association with the generation of the location data. The user identification data may comprise one or more of: image data, video data, biometric data (e.g. fingerprint, DNA, retinal scan, etc. data), or any other type of data that may be used to verify the identity of the user at or near the time the location data is generated. And the user identification sensor may comprise one or more of: a camera, microphone, heat sensor, biometric data sensor, or any other type of device capable of sensing/generating the aforementioned types of user identification data.

The user identification data is wirelessly transmitted in association with the location data and/or medication intake data to central monitoring station 160 via a wireless transmitter. The user identification data is stored in association with the location data and/or medication intake data by central monitoring station 160 and is retrievable therefrom by an authorized monitor, such as a parent, guardian, parole officer, court liaison, spouse, friend, or other authorized group or individual. In some cases, the monitor is able to retrieve the location data via a network connected user interface device communicatively coupled—via the network—to central monitoring station 160 and/or to user detached monitor device 120. The user identification data may be transmitted to central monitoring station 160 independent of the aforementioned medication intake monitoring data and/or user location data, for example, during a periodic check-in with central monitoring system 160.

User detached monitor device 120 may further comprise a memory communicatively coupled to a control unit—which is also communicatively coupled to the location sensor, the identification sensor, the medication monitoring module, and the wireless transceiver—for controlling the operations thereof in accordance with the functionalities described herein. The memory may include non-transient instructions (e.g., software of firmware based instructions) executable by the control unit to perform and/or enable various functions associated with user detached monitor device 120. Such functions may include, but are not limited to, the function of the medication monitoring module, the location circuitry, and/or the user identification circuitry. As user detached monitor device 120 is portable, each of the components may be located within, immediately adjacent to, or exposed without, a device housing whose dimensions are such that user detached monitor device 120 as a whole may be discretely carried by the user, for example, within a pocket or small purse.

Central monitoring station 160 in some cases includes a server supported website, which may be supported by a server system comprising one or more physical servers, each having a processor, a memory, an operating system, input/output interfaces, and network interfaces, all known in the art, coupled to the network. The server supported website comprises one or more interactive web portals through which the monitor may monitor the location of the user in accordance with the described embodiments. In particular, the interactive web portals may enable the monitor to retrieve the location and user identification data of one or more users, set or modify ‘check-in’ schedules, and/or set or modify preferences. The interactive web portals are accessible via a personal computing device, such as for example, a home computer, laptop, tablet, and/or smart phone.

In some embodiments, the server supported website comprises a mobile website accessible via a software application on a mobile device (e.g. smart phone). The mobile website may be a modified version of the server supported website with limited or additional capabilities suited for mobile location monitoring.

In addition, medication intake monitoring system 100 includes a historical database 1061 that maintains various of the data transferred from user detached monitor device 120 and/or derivatives of such information including, for example, any notes provided by a monitoring officer and/or results generated by processing of the information received from user detached monitor device 120. In some cases, the data is organized in relation to the monitored individual to whom the data pertains. Based upon the disclosure provided herein, one of ordinary skill in the art will perceive of a variety of information that may be maintained in historical database 1061.

Turning to FIG. 1b, a block diagram of user detached monitor device 120 is shown in accordance with one or more embodiments. User detached monitor device 120 includes wireless transceiver circuitry 128 that is capable of sending and receiving information via wireless link 133 to/from wide area wireless network 150. Wireless transceiver circuitry 128 may be any circuitry, integrated circuit, processor or controller, or combination thereof that is capable of supporting wireless communication. Such wireless communication may include, but is not limited to, cellular telephone communication, Internet communication via a Wi-Fi access point, or both. In addition, user detached monitor device 120 includes a vibrator 112, a speaker 114, and a visual display and touch screen 116. In some cases, at scheduled times a monitored individual associated with user detached monitor device 120 is alerted of a need to check-in or to take a medication. The schedule of check-in times and/or medication times may be downloaded to a memory 124 by central monitoring station 160 via wireless link 133. The monitored individual may be alerted by one or more of: a visual prompt via visual display and touch screen 116, an audio prompt via speaker 114, and a tactile prompt via vibrator 112. Each of vibrator 112, speaker 114, and visual display and touch screen 116 is communicatively coupled to memory 124 and/or a control circuit 122 for controlling the operations thereof. In some cases, control circuit 122 includes a processor. In various cases, control circuit 122 is part of an integrated circuit. In one or more cases, memory 124 is included in an integrated circuit with control circuit 122. In various cases, memory 124 may include non-transient instructions (e.g., software of firmware based instructions) executable by controller circuit 122 to perform and/or enable various functions associated with user detached monitor device 120. A visual prompt may include, but is not limited to, text, images and/or a combination thereof, or a series of such visual prompts. An audio prompt may include, but is not limited to, one or more different audio prompts, or a series thereof. Each prompt may be stored in memory 124 and retrieved in accordance with the schedule that is also maintained in memory 124. In some embodiments, alerting the monitored individual involves a prompt that includes an e-mail or text message generated by central monitoring station 160 (e.g. the server supported website) and transmitted to the e-mail account or cellular phone number corresponding to user detached monitor device 120. In particular embodiments, such a prompt may include a ‘post’ on the user's ‘wall,’ ‘feed,’ or other social networking privilege. In some embodiments, the prompt may comprise an automated or live phone call to the user.

User detached monitor device 120 further includes a medication monitoring module 178. Medication monitoring module 178 operates to request and process information corresponding to the taking of medication by the individual associated with user detached monitor device 120. The requests are made via one or more of visual display and touch screen 116, speaker 114, and/or vibrator 112. In some embodiments, medication monitoring module 178 causes a message to be displayed via visual display and touch screen 116 to the individual associated with user detached monitor device 120 indicating a medication that is to be taken. The processes are controlled by controller circuit 122 executing commands corresponding to medication monitoring module 178.

Medication monitoring module 178 waits for a response from the monitored individual that they are ready to take the medication. The response may include, but is not limited to, the selection of a radio button displayed on visual display and touch screen 116. When the response is received, a camera 173 of user detached monitor device 120 is used to record the type of medication being taken followed by recording an image of the medication in the monitored individual's mouth, followed by subsequent image of the individual's mouth with the tongue down after the medication is swallowed, and then followed by a subsequent image of the individuals mouth with the tongue up after the medication is swallowed. All of these images can be shared with a monitoring officer either in real time or stored for later review. In some embodiments, the medication monitoring module includes image processing software operable to discern the medication placed in the monitored individual's mouth from the image taken of the mouth, and then later can discern that the medication is no longer in the monitored individual's mouth based upon the subsequent two images with the tongue up and the tongue down, respectively. FIGS. 2a-2c discussed in detail below show one embodiment of processes performed by a medication monitoring module.

User detached monitor device 120 further includes user identification circuitry 179 capable of gathering user identification information from one or more of a microphone 171, camera 173, a temperature sensor 175, and/or a biometric sensor 177. In some cases, user identification circuitry 179 is incorporated in an integrated circuit with control circuit 122. Microphone 171 is capable of accurately capturing the sound of a monitored individual's voice, camera 173 is capable of accurately capturing images including, for example, an image of the monitored individual's face, temperature sensor 175 is capable of accurately capturing an ambient temperature around user detached monitor device 120, and biometric sensor 177 is capable of accurately capturing biometric data about the user including, but not limited to, a thumb print, a retinal scan, or a breath-based alcohol measurement. Based upon the disclosure provided herein, one of ordinary skill in the art will recognize a variety of biometric data and corresponding sensors that may be used in relation to different embodiments. Under the direction of control circuitry 122, user identification circuitry 179 assembles one or more elements of data gathered by microphone 171, camera 173, temperature sensor 175, and/or biometric sensor 177 into a user identification package which is forwarded to central monitoring station 160 via wireless transceiver circuitry 128.

User detached monitor device 120 additionally includes location circuitry 126. Location circuitry 126 may include one or more of, a GPS processing circuit capable of fixing a location of user detached monitor device 120 using GPS data, and/or a cell tower triangulation processing circuit capable of fixing a location of user detached monitor device 120 using cell tower triangulation data.

Turning to FIGS. 2a-2c, flow diagrams 290, 292, 294 show a method in accordance with some embodiments for monitoring medication intake by a monitored individual. Following flow diagram 290 of FIG. 2a, client medication, dosage, usage, and refill reminders are loaded into a client database (block 200). This may be done, for example, by a monitoring officer using remote monitoring interface 185. Alternatively, this information may be automatically updated from a medical provider or pharmacy of the monitored individual. In some embodiments, this client database may be maintained as part of central monitoring station 160, and the entered data may be maintained as part of historical database 1061. In addition, a client calendar is added to the client database which, as mentioned before, may be maintained as part of historical database 1061 (block 202).

In some embodiments, the aforementioned client calendar and medication information may be downloaded from central monitoring station 160 to user detached monitor device 120 via wide area wireless network 150. In other embodiments, the aforementioned client calendar and medication information is not downloaded, but rather only reminders based upon this information are downloaded to a client device (in some cases the client device is user detached monitor device 120).

Based upon the client calendar, it is determined whether it is time for a monitored individual to take a required medication (block 204). In some cases, this is done by repeatedly comparing the next date in the calendar with a clock running on central monitoring station 160 where central monitoring station 160 is responsible for issuing a notice to take the medication, or by repeatedly comparing the next date in the calendar with a clock running on the client device where the client device is responsible for issuing a notice to take the medication. Based upon the disclosure provided herein, one of ordinary skill in the art will recognize a variety of ways in which a determination of when to issue a notice to take a medication may be done in accordance with different embodiments.

Where it is determined that it is time for the client to take a medication (block 204), an incremental reminder is sent to the monitored individual via the client device (block 206). Such incremental notices may begin some preset time period before the medication is to be taken (e.g., three hours, one hour, fifteen minutes, or the like) and are repeated at a preselected frequency (e.g., every ten minutes) until the individual either takes the medication or the time to take the medication expires. The incremental notices may, for example, be provided to the monitored individual as a text message via visual display and touch screen 116, as an audio message via speaker 114, or as a tactile message via vibrator 112, or some combination of the aforementioned. It is determined whether the monitored individual accepted the notice (block 208). Where, for example, the client accepts the notice by touching a radio button on visual display and touch screen 116, determining whether the client accepted the notice is done by determining whether the radio button was selected by the monitored individual. Based upon the disclosure provided herein, one of ordinary skill in the art will recognize a variety of mechanisms that may be used by a monitored individual to accept a notice to take a medication and corresponding processes to determine whether the notice was accepted.

Where the monitored individual does not accept the notice within a defined time period (block 212 and block 208), it is determined whether it was the final notice for the particular medication period (block 210). A final notice may be, for example, the fourth of four notices spaced fifteen minutes apart over the course of an hour. Where it is not the final notice (block 210), the process returns to block 204 to await the next incremental notice of block 206. Alternatively, where it was the final notice for the particular medication period (block 210), a monitoring officer is sent an alert of a missed medication (block 214). This alert may be sent to central monitoring station 160 and include an identification of the monitored individual, the medication missed, and the time when the medication was missed.

Alternatively, where the monitored individual does accept the notice within a defined time period (block 212 and block 208), a message is provided to the monitored individual via the client device indicating which medication is to be taken and the dosage for the medication, and the monitored individual selects the medication they intend to take (block 216). In some embodiments, this includes providing a written message via visual display and touch screen 116 where the message has indicates one or more medications each having a radio button by the indicated medication. In response to the message, the monitored individual presses the radio button of one of the presented medications indicating that that is the medication they intend to take.

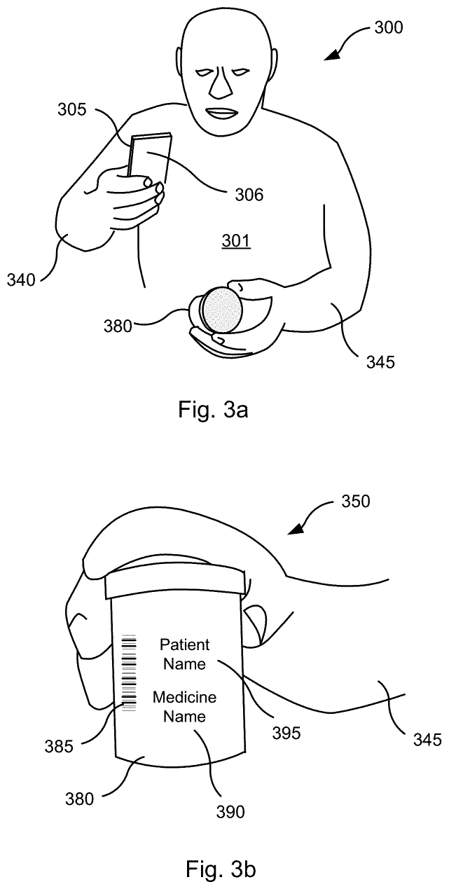

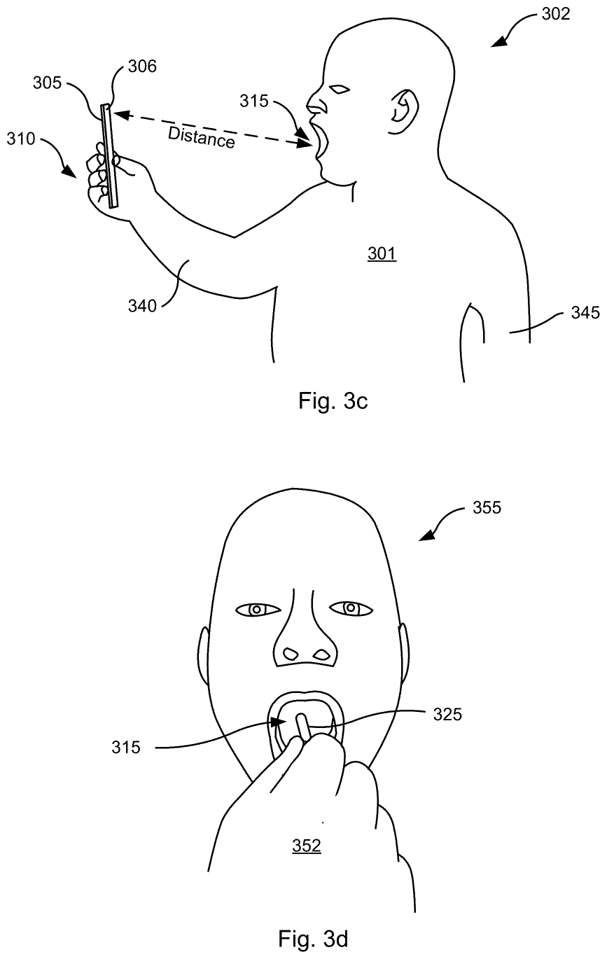

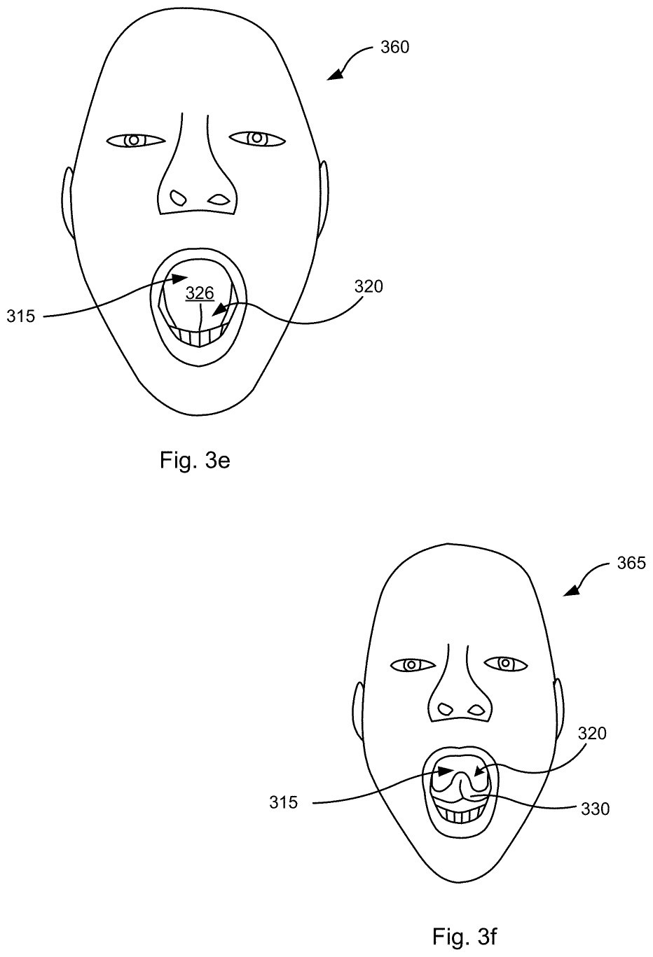

A video camera on the client device is enabled such that when looking at visual display and touch screen 116 of the client device (block 218). The monitored individual is prompted to hold the medication in front of the camera so that it can be seen on the display of the client device (block 220). Turning to FIG. 3a, a graphical depiction 300 shows a monitored individual 301 holding a client device 305 with a camera opening 306 in their right arm 340 while holding a medication container 380 in their right arm 345 such that a camera in client device 305 captures an image of medication container 380.

Returning to FIG. 2a, an image of a medication container holding the selected medication is captured (block 221). The image includes, for example, the name of the medication and dosage of each pill in the container and/or a bar code that when decoded includes the aforementioned information. In some cases, the image is captured once the barcode and medication name can be seen within a frame captured by the camera. In other cases, the monitored individual manually selects image capture once they are satisfied that the image includes the relevant information. Turning to FIG. 3b, a graphical depiction 350 shows an image captured by client device 305 of medication container 380. As shown, the image includes medication container 380 showing a bar code 385, a patient name 395, and a medicine name 390.

Turning to FIG. 2b and following flow diagram 292, based upon the image of the medication container it is determined whether the medication matches that selected in block 216 (block 222). In some cases this is done automatically by either using the barcode or the medication name captured in the image to compare with the medication selected in block 216. In other cases, a monitoring officer looks at the captured image and makes a determination that the medication in the image matches that selected in block 216. Based upon the disclosure provided herein, one of ordinary skill in the art will recognize other methods for determining that the correct medication is presented.

Returning to FIG. 2b, where it is determined that either the medication container shown in the image is not correct or that the image of the medication container does not properly show the information needed to make the determination (block 222), the monitored individual is alerted of the error (block 224) and the process is returned to block 208. This alert may be, but is not limited to, a written message provided via visual display and touch screen 116 of the client device. Alternatively, where the correct medication is selected (block 222), the monitored individual is prompted to orient the client device to take an image of their open, empty mouth (block 226). This prompt may be, but is not limited to, a written message provided via visual display and touch screen 116 of the client device. To do this, the monitored individual holds the client device such that the camera of the client device is aimed at their mouth. Turning to FIG. 3c, a graphical depiction 302 shows monitored individual 301 holding client device 305 in their hand 310 a distance from their open mouth 315 such that camera 306 is aimed at open mouth 315. Returning to FIG. 2b, an image of the open mouth is captured by the client device (block 228). In some cases, the image is captured once the open mouth is fully in view of the camera. In other cases, the monitored individual manually selects image capture once they are satisfied that the image includes the open mouth. Turning to FIG. 3e, a graphical depiction 360 shows an image captured by client device 305 of the open mouth 315 including top 326 of the tongue 320 of monitored individual 301.

Returning to FIG. 2b, the monitored individual is prompted to take a video of them placing the medication into their open mouth (block 230). This process includes holding the client device in one hand similar to that discussed above in relation to block 226, and placing the medication in the open mouth using their other hand while a video of the medication placement is captured (block 232). In some cases, the video begins once the other hand holding the medication enters the frame of view and continues until the end of the process. In other cases, the monitored individual selects video capture once they are satisfied that the image includes the open mouth and maintains the video capture while they place the medication in their mouth and subsequent ingestion and proof of ingestion. Turning to FIG. 3d, a graphical depiction 352 of one frame of the captured video is shown with a hand 352 of the monitored individual within the frame of view and placing a medication 325 in open mouth 315.

Returning to FIG. 2b, once the hand has left the frame of view, frames of the video are searched to determined whether the medication remained on the tongue of the monitored individual (block 234). In some embodiments, this is done by comparing the frame of the video after the hand has left the frame of view with the earlier captured image of the open, empty mouth. Differences in the images are identified and it is determined whether the differences correspond to an element approximately the size of the selected medication. As another example, a monitoring officer may watch the video of the medication being placed in the mouth to make the determination. Based upon the disclosure provided herein, one of ordinary skill in the art will recognize other approaches that may be used to determine whether the medication is in the mouth of the monitored individual.

Where it is determined that the medication was not left behind on the tongue of the monitored individual (block 234), the monitored individual is alerted of the error (block 236) and the process is returned to block 208. Alternatively, where the medication is determined to be in the monitored individual's mouth (block 234), the monitored individual is prompted to continue the video while they swallow the medication (block 238).

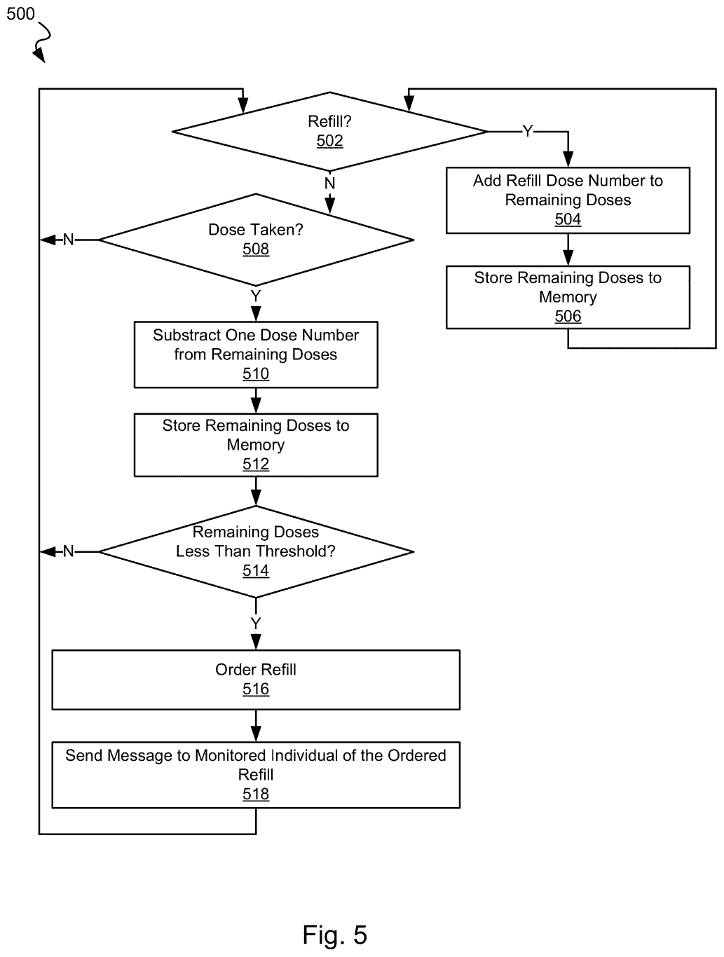

Turning to FIG. 2c, it is determined whether the swallowing process has occurred and that the medication has thus been taken (block 240). In some cases, this may be determined automatically by using frames form the video to demonstrate that the mouth has closed and re-opened. In other cases, this is determined by a monitoring officer watching the video. Based upon the disclosure provided herein, one of ordinary skill in the art will recognize other methods for determining that the swallowing process has completed. Where the swallowing process has not yet occurred (block 240), it is determined whether a timeout condition has occurred (block 242). This timeout occurs when more than a predefined time period passes from when the monitored individual is prompted to swallow the medication until the medication is swallowed. Where a timeout condition occurs (block 242), a message is sent to the monitoring officer indicating a failure (block 234).

Where it is determined that the medication was swallowed (block 240), the monitored individual is prompted to open their mouth while the video continues to show their mouth with the tongue in a down position (block 246). Turning to FIG. 3e, graphical depiction 360 shows an image captured by client device 305 of the open mouth 315 including a top 326 of the tongue 320 of monitored individual 301 showing that the medication has cleared the monitored individual's mouth. It is determined whether the medication remains in the mouth of the monitored individual (block 248). In some embodiments, this is done by comparing the frame of the video showing the open mouth and comparing it with the earlier captured image of the open, empty mouth. Differences in the images are identified and it is determined whether the differences correspond to an element approximately the size of the selected medication. As another example, a monitoring officer may watch the video of the medication being placed in the mouth to make the determination. Based upon the disclosure provided herein, one of ordinary skill in the art will recognize other approaches that may be used to determine whether the medication is in the mouth of the monitored individual. Where the medication has not yet been determined to have cleared the monitored individual's mouth (block 248), it is determined whether a timeout condition has occurred (block 250). This timeout occurs when more than a predefined time period passes from when the monitored individual is prompted to swallow the medication until the medication is swallowed. Where a timeout condition occurs (block 250), a message is sent to the monitoring officer indicating a failure (block 252).

Where it is determined that the medication has cleared the monitored individual's mouth (block 248), the monitored individual is prompted to open their mouth while the video continues to show their mouth with the tongue in an up position to show that the medication is not under the tongue of the monitored individual (block 254). Turning to FIG. 3f, graphical depiction 365 shows an image captured by client device 305 of the open mouth 315 including the tongue 320 of monitored individual 301 showing an underside 330 of the tongue 320 to demonstrate that the medication has cleared the monitored individual's mouth. It is determined whether the medication remains in the mouth of the monitored individual (block 256). In some embodiments, this is done by comparing the frame of the video showing the open mouth and looking for anything in the mouth that is approximately the size of the medication. As another example, a monitoring officer may watch the video of the medication being placed in the mouth to make the determination. Based upon the disclosure provided herein, one of ordinary skill in the art will recognize other approaches that may be used to determine whether the medication is in the mouth of the monitored individual. Where the medication has not yet been determined to have cleared the monitored individual's mouth (block 256), it is determined whether a timeout condition has occurred (block 258). This timeout occurs when more than a predefined time period passes from when the monitored individual is prompted to swallow the medication until the medication is swallowed. Where a timeout condition occurs (block 258), a message is sent to the monitoring officer indicating a failure (block 260). Alternatively, where it is determined that the medication has cleared the monitored individual's mouth (block 256), both the monitored individual and the monitoring officer are alerted that the process has completed successfully (block 262). Where the known dose of medication was taken (block 262), in some embodiments (for this reason it is shown in dashed lines) the medication status is updated (block 264). Such a medication status update includes calculating a number of remaining doses, and where appropriate automatically ordering refills and sending a pickup reminder to the monitored individual. FIG. 5 below shows one approach to medication status updating that may be used in relation to different embodiments.

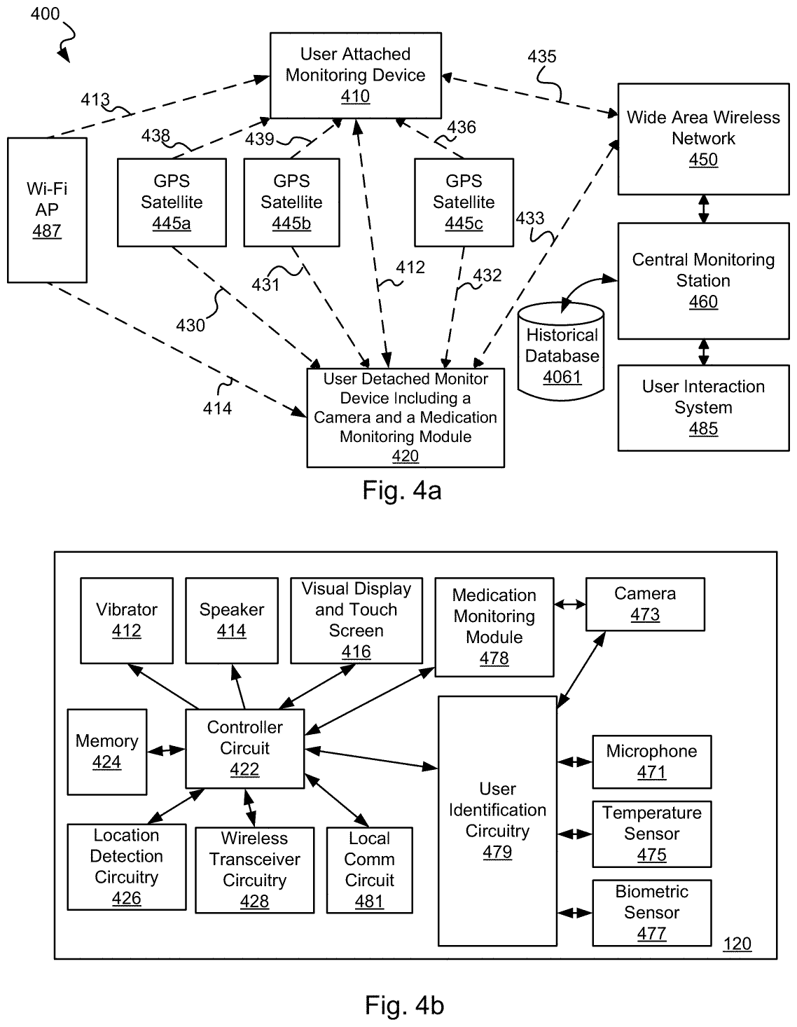

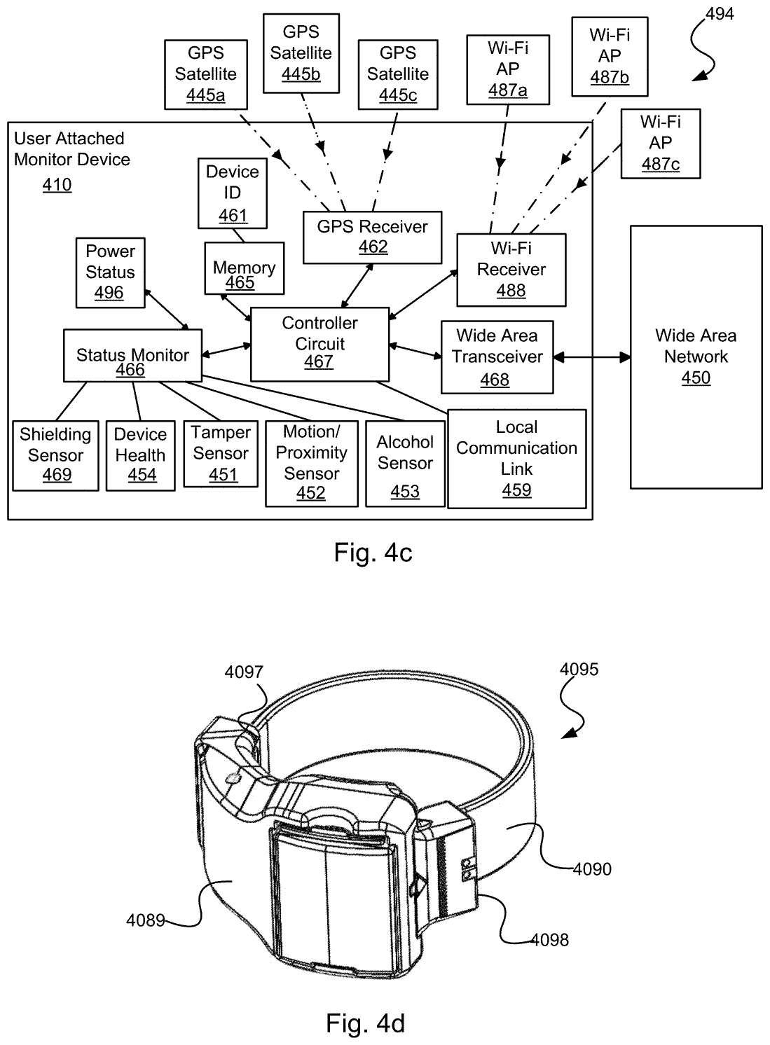

Turning to FIG. 4a, a block diagram illustrates a hybrid medication intake monitoring system 400 including client device that comprises both a user detached monitor device 420 and a user attached monitor device 410 in accordance with various embodiments. A local communication link 412 allows for communication between user attached monitor device 410 and user detached monitor device 420. Local communication link 412 may be any communication link that is capable of transferring information or otherwise communicating between two devices within a relatively short distance of each other. In some cases, for example, local communication link 412 may be a BlueTooth™ communication link. In other examples, local communication link 412 may be a line of sight infared communication link. As yet other examples, local communication link 412 may be a WiFi communication link. Based upon the disclosure provided herein, one of ordinary skill in the art will recognize a variety of communication protocols and/or media that may be used to implement local communication link 412.

User detached monitor device 420 includes a medication monitoring module operable to enable monitoring of a medication by an individual associated with user detached monitor device 420. User detached monitor device 420 is portable, and may be any device that is recognized as being used by or assigned to an individual being monitored, but is not physically attached to the individual being monitored by a tamper evident attaching device. User detached monitor device 420 may be, but is not limited to, a mobile telephone. A central monitoring station 460 is wirelessly coupled to user detached monitor device 420 via one or more wide area wireless (e.g., cellular telephone network, Internet via a Wi-Fi access point, or the like) communication networks 450.

User detached monitor device 420 includes a medication monitoring module that operates to request and process information corresponding to the taking of medication by the individual associated with user detached monitor device 420. In some embodiments, the medication monitoring module causes a message to be displayed to the individual associated with user detached monitor device 420 indicating a medication that is to be taken. The medication monitoring module waits for a response from the monitored individual that they are ready to take the medication. The camera of user detached monitor device 420 is used to record the type of medication being taken followed by recording an image of the medication in the monitored individual's mouth, followed by subsequent image of the individual's mouth with the tongue down after the medication is swallowed, and then followed by a subsequent image of the individuals mouth with the tongue up after the medication is swallowed. All of these images can be shared with a monitoring officer either in real time or stored for later review. In some embodiments, the medication monitoring module includes image processing software operable to discern the medication placed in the monitored individual's mouth from the image taken of the mouth, and then later can discern that the medication is no longer in the monitored individual's mouth based upon the subsequent two images with the tongue up and the tongue down, respectively. FIGS. 2a-2c discussed in detail below show one embodiment of processes performed by a medication monitoring module.

The aforementioned medication intake monitoring data and/or other data gathered by user detached monitor device 420 is wirelessly transmitted to central monitoring station 460 via wide area wireless network 450 accessed via a wireless link 433. Central monitoring station 460 may be any location, device or system where the location data is received, including by way of non-limiting example: a cellular/smart phone, an email account, a website, a network database, and a memory device. The location data is stored by central monitoring station 460 and is retrievable therefrom by a monitor, such as a parent, guardian, parole officer, court liaison, spouse, friend, or other authorized group or individual. In this manner, monitor is able to respond appropriately to the detected out-of-bounds activity by a user. In some cases, the monitor is able to retrieve the location data via a user interaction system 485 which may be, but is not limited to, a network connected user interface device communicatively coupled via a network to central monitoring station 460 and/or directly to user detached monitor device 420 via wide area wireless network 450.

Additionally, user detached monitor device 420 may include a location sensor that senses the location of the device and generates a location data. The location data may comprise one or more of: global positioning system (“GPS”) data, Assisted GPS (“A-GPS”) data, Advanced Forward Link Trilateration (“AFLT”) data, and/or cell tower triangulation data. Where GPS is used, user detached monitor device 420 receives location information from three or more GPS satellites 445a, 445b, 445c via respective communication links 430, 431, 432. The aforementioned location data is utilized verify the location of a user associated with user detached monitor device 420 at various points as more fully discussed below. User detached monitor device 420 is considered “ambiguous” because it is not attached to the user in a tamper resistant/evident way, but rather is freely severable from the user and thus could be used by persons other than the target. Various processes discussed herein mitigate the aforementioned ambiguity to yield a reasonable belief that information derived from user detached monitor device 420 corresponds to the target.

The location data and/or other data gathered by user detached monitor device 420 is wirelessly transmitted to central monitoring station 460 via wide area wireless network 450 accessed via a wireless link 433. Central monitoring station 460 may be any location, device or system where the location data is received, including by way of non-limiting example: a cellular/smart phone, an email account, a website, a network database, and a memory device. The location data is stored by central monitoring station 460 and is retrievable therefrom by a monitor, such as a parent, guardian, parole officer, court liaison, spouse, friend, or other authorized group or individual. In this manner, monitor is able to respond appropriately to the detected out-of-bounds activity by a user. In some cases, the monitor is able to retrieve the location data via a user interaction system 485 which may be, but is not limited to, a network connected user interface device communicatively coupled via a network to central monitoring station 460 and/or directly to user detached monitor device 420 via wide area wireless network 450.

User detached monitor device 420 may further include a user identification sensor operable to generate user identification data for identifying the user in association with the generation of the location data. The user identification data may comprise one or more of: image data, video data, biometric data (e.g. fingerprint, DNA, retinal scan, etc. data), or any other type of data that may be used to verify the identity of the user at or near the time the location data is generated. And the user identification sensor may comprise one or more of: a camera, microphone, heat sensor, biometric data sensor, or any other type of device capable of sensing/generating the aforementioned types of user identification data.

The user identification data is wirelessly transmitted in association with the location data to central monitoring station 460 via a wireless transmitter communicatively coupled to the user identification sensor. The user identification data is stored in association with the location data by central monitoring station 460 and is retrievable therefrom by a monitor, such as a parent, guardian, parole officer, court liaison, spouse, friend, or other authorized group or individual. In some cases, the monitor is able to retrieve the location data via a network connected user interface device communicatively coupled—via the network—to central monitoring station 460 and/or to user detached monitor device 420. The location data may be transmitted to central monitoring station 460 independent of the user identification data, for example, during a periodic check-in with central monitoring system 460.

User detached monitor device 420 may further comprise a memory communicatively coupled to a control unit—which is also communicatively coupled to the location sensor, the identification sensor and the wireless transceiver—for controlling the operations thereof in accordance with the functionalities described herein. The memory may include non-transient instructions (e.g., software of firmware based instructions) executable by the control unit to perform and/or enable various functions associated with user detached monitor device 420. As user detached monitor device 420 is portable, each of the components may be located within, immediately adjacent to, or exposed without, a device housing whose dimensions are such that user detached monitor device 420 as a whole may be discretely carried by the user, for example, within a pocket or small purse. User detached monitor device 420 may include a Wi-Fi transceiver capable of receiving information from one or more Wi-Fi access points 487 that can be used to discern location via a Wi-Fi communication link 414.

Central monitoring station 460 preferably comprises a server supported website, which may be supported by a server system comprising one or more physical servers, each having a processor, a memory, an operating system, input/output interfaces, and network interfaces, all known in the art, coupled to the network. The server supported website comprises one or more interactive web portals through which the monitor may monitor the location of the user in accordance with the described embodiments. In particular, the interactive web portals may enable the monitor to retrieve the location and user identification data of one or more users, set or modify ‘check-in’ schedules, and/or set or modify preferences. The interactive web portals are accessible via a personal computing device, such as for example, a home computer, laptop, tablet, and/or smart phone.

In some embodiments, the server supported website comprises a mobile website accessible via a software application on a mobile device (e.g. smart phone). The mobile website may be a modified version of the server supported website with limited or additional capabilities suited for mobile location monitoring.

User attached monitor device 410 includes a location sensor that senses the location of the device and generates a location data. The location data may comprise one or more of: global positioning system (“GPS”) data, Assisted GPS (“A-GPS”) data, Advanced Forward Link Trilateration (“AFLT”) data, and/or cell tower triangulation data. Where GPS is used, user attached monitor device 410 receives location information from three or more GPS satellites 445a, 445b, 445c via respective communication links 436, 438, 439. The aforementioned location data is utilized to verify the location of a user associated with user attached monitor device 410 at various points as more fully discussed below. User attached monitor device 410 is considered “non-ambiguous” because it is physically attached to the user in a tamper resistant way and as such provides information only about the user to which it is attached.

The location data and/or other data gathered by user attached monitor device 410 is wirelessly transmitted to central monitoring station 460 via a wide area wireless network 450 accessed via a wireless link 435. Central monitoring station 460 may be any location, device or system where the location data is received, including by way of non-limiting example: a cellular/smart phone, an email account, a website, a network database, and a memory device. The location data is stored by central monitoring station 460 and is retrievable therefrom by a monitor, such as a parent, guardian, parole officer, court liaison, spouse, friend, or other authorized group or individual. In this manner, monitor is able to respond appropriately to the detected out-of-bounds activity by a user.

User attached monitor device 410 may further comprise a memory communicatively coupled to a control unit—which is also communicatively coupled to the location sensor, the identification sensor and the wireless transceiver—for controlling the operations thereof in accordance with the functionalities described herein. The memory may include non-transient instructions (e.g., software of firmware based instructions) executable by the control unit to perform and/or enable various functions associated with user attached monitor device 410. User attached monitor device may include a strap which can be wrapped around a limb of the individual being monitored to secure user attached monitor device to the individual. The strap includes one or more tamper circuits and/or sensors that allow for a determination as to whether the device has been removed or otherwise tampered. Examples of a strap and tamper detection circuitry that may be used in relation to various embodiments discussed herein are described in U.S. Pat. No. 9,355,579 entitled “Methods for Image Based Tamper Detection”, and filed by Buck et al. on Sep. 45, 2014; and US Pat. Pub. No. US 2017-0270778 A1 entitled “Systems and Methods for Improved Monitor Attachment”, and filed by Melton et al. on Mar. 21, 2016. Both of the aforementioned references are incorporated herein by reference for all purposes. Based upon the disclosure provided herein, one of ordinary skill in the art will recognize a variety of straps, tamper circuits, tamper devices, and/or attachment and tamper detection approaches that may be used in relation to various embodiments. User attached monitor device 410 may include a Wi-Fi transceiver capable of receiving information from one or more Wi-Fi access points 487 that may be used to identify location via a Wi-Fi communication link 413.

In addition, medication intake monitoring system 400 includes a historical database 4061 that maintains various of the data transferred from user detached monitor device 420 and/or derivatives of such information including, for example, any notes provided by a monitoring officer and/or results generated by processing of the information received from user detached monitor device 420. In some cases, the data is organized in relation to the monitored individual to whom the data pertains. Based upon the disclosure provided herein, one of ordinary skill in the art will perceive of a variety of information that may be maintained in historical database 4061.

Turning to FIG. 4b, a block diagram of user detached monitor device 420 is shown in accordance with one or more embodiments. User detached monitor device 420 includes wireless transceiver circuitry 428 that is capable of sending and receiving information via wireless link 433 to/from wide area wireless network 450. Wireless transceiver circuitry 428 may be any circuitry, integrated circuit, processor or controller, or combination thereof that is capable of supporting wireless communication. Such wireless communication may include, but is not limited to, cellular telephone communication, Internet communication via a Wi-Fi access point, or both. In addition, user detached monitor device 420 includes a vibrator 412, a speaker 414, and a visual display and touch screen 416. In some cases, at scheduled times a user of user detached monitor device 420 is alerted of a need to check-in or to take a medication. The schedule of check-in times may be downloaded to a memory 424 by central monitoring station 460 via wireless link 433. The user may be alerted by one or more of: a visual prompt via visual display and touch screen 416, an audio prompt via speaker 414, and a tactile prompt via vibrator 412. Each of vibrator 412, speaker 414, and visual display and touch screen 416 is communicatively coupled to memory 424 and/or a control circuit 422 for controlling the operations thereof. In some cases, control circuit 422 includes a processor. In various cases, control circuit 422 is part of an integrated circuit. In one or more cases, memory 424 is included in an integrated circuit with control circuit 422. In various cases, memory 424 may include non-transient instructions (e.g., software of firmware based instructions) executable by controller circuit 422 to perform and/or enable various functions associated with user detached monitor device 420. A visual prompt may include, but is not limited to, text, images and/or a combination thereof, or a series of such visual prompts. An audio prompt may include, but is not limited to, one or more different audio prompts, or a series thereof. Each prompt may be stored in memory 424 and retrieved in accordance with the schedule that is also maintained in memory 424. In some embodiments, alerting the user involves a prompt that includes an e-mail or text message generated by central monitoring station 460 (e.g. the server supported website) and transmitted to the e-mail account or cellular phone number corresponding to user detached monitor device 420. In particular embodiments, such a prompt may include a ‘post’ on the user's ‘wall,’ ‘feed,’ or other social networking privilege. In some embodiments, the prompt may comprise an automated or live phone call to the user.

User detached monitor device 420 further includes medication monitoring module 478. Medication monitoring module 478 operates to request and process information corresponding to the taking of medication by the individual associated with user detached monitor device 420. The requests are made via one or more of visual display and touch screen 416, speaker 414, and/or vibrator 412. In some embodiments, medication monitoring module 478 causes a message to be displayed via visual display and touch screen 416 to the individual associated with user detached monitor device 420 indicating a medication that is to be taken. The processes are controlled by controller circuit 422 executing commands corresponding to medication monitoring module 478.

Medication monitoring module 478 waits for a response from the monitored individual that they are ready to take the medication. The response may include, but is not limited to, the selection of a radio button displayed on visual display and touch screen 416. When the response is received, camera 473 of user detached monitor device 420 is used to record the type of medication being taken followed by recording an image of the medication in the monitored individual's mouth, followed by subsequent image of the individual's mouth with the tongue down after the medication is swallowed, and then followed by a subsequent image of the individuals mouth with the tongue up after the medication is swallowed. All of these images can be shared with a monitoring officer either in real time or stored for later review. In some embodiments, the medication monitoring module includes image processing software operable to discern the medication placed in the monitored individual's mouth from the image taken of the mouth, and then later can discern that the medication is no longer in the monitored individual's mouth based upon the subsequent two images with the tongue up and the tongue down, respectively. FIGS. 2a-2c discussed in detail below show one embodiment of processes performed by a medication monitoring module.

User detached monitor device 420 further includes user identification circuitry 479 capable of gathering user identification information from one or more of a microphone 471, a camera 473, a temperature sensor 475, and/or a biometric sensor 477. In some cases, user identification circuitry 479 is incorporated in an integrated circuit with control circuit 422. Microphone 471 is capable of accurately capturing the sound of a user's voice, camera 473 is capable of accurately capturing images including, for example, an image of the user's face, temperature sensor 475 is capable of accurately capturing an ambient temperature around user detached monitor device 420, and biometric sensor 477 is capable of accurately capturing biometric data about the user including, but not limited to, a thumb print, a retinal scan, or a breath-based alcohol measurement. Based upon the disclosure provided herein, one of ordinary skill in the art will recognize a variety of biometric data and corresponding sensors that may be used in relation to different embodiments. Under the direction of control circuitry 422, user identification circuitry 479 assembles one or more elements of data gathered by microphone 471, a camera 473, a temperature sensor 475, and/or a biometric sensor 477 into a user identification package which is forwarded to central monitoring station 460 via wireless transceiver circuitry 428.

User detached monitor device 420 additionally includes location circuitry 426. Location circuitry 426 may include one or more of, a GPS processing circuit capable of fixing a location of user detached monitor device 420 using GPS data, and/or a cell tower triangulation processing circuit capable of fixing a location of user detached monitor device 420 using cell tower triangulation data. A local communication link 481 controls communication between user detached monitor device 420 and user attached monitor device 410. In some embodiments, local communication link 481 supports a BlueTooth™ communication protocol and is capable of both receiving information from user attached monitor device 410 and transmitting information to user attached monitor device 410. In other embodiments, Local communication link 481 supports a Wi-Fi communication protocol and is capable of both receiving information from user attached monitor device 410 and transmitting information to user attached monitor device 410. In some cases, local communication link 481 supports communication in only a receive or transmit direction. Based upon the disclosure provided herein, one of ordinary skill in the art will recognize a variety of communication protocols and information transfer directions that may be supported by local communication link 481 in accordance with different embodiments.

Turning to FIG. 4c, a block diagram 494 of user attached monitor device 410 including a local communication link 459 in accordance with some embodiments. Local communication link 459 controls communication between user attached monitor device 410 and user detached monitor device 420. In some embodiments, local communication link 459 supports a BlueTooth™ communication protocol and is capable of both receiving information from user detached monitor device 420 and transmitting information to user detached monitor device 420. In other embodiments, local communication link 459 supports a Wi-Fi communication protocol and is capable of both receiving information from user detached monitor device 410 and transmitting information to user detached monitor device 410. In some cases, local communication link 459 supports communication in only a receive or transmit direction. Based upon the disclosure provided herein, one of ordinary skill in the art will recognize a variety of communication protocols and information transfer directions that may be supported by local communication link 459 in accordance with different embodiments.

As shown, user attached monitor device 410 includes a device ID 461 that may be maintained in a memory 465, and thus is accessible by a controller circuit 467. Controller circuit 467 is able to interact with a GPS receiver 462 and memory 465 at times for storing and generating records of successively determined GPS locations. Similarly, controller circuit 467 is able to interact with a Wi-Fi receiver 488 and memory 465 at times for storing and generating records of successively determined Wi-Fi access point identifications and signal strength. In some cases, memory 465 may include non-transient instructions (e.g., software of firmware based instructions) executable by controller circuit 467 to perform and/or enable various functions associated with user attached monitor device 410. As user attached monitor device 410 comes within range of one or more Wi-Fi access points (e.g., Wi-Fi access points 487), Wi-Fi receiver 488 senses the signal provided by the respective Wi-Fi access points, and provides an identification of the respective Wi-Fi access point and a signal strength of the signal received from the Wi-Fi access point to Wi-Fi receiver 488. This information is provided to controller circuit 467 which stores the information to memory 465.

Where user attached monitor device 410 is operating in a standard mode, controller circuit 467 causes an update and reporting of the location of user attached monitor device 410 via a cellular transceiver 468 and a wide area communication network 450 in accordance with a first time period. In contrast, where user attached monitor device 410 is within range of a public Wi-Fi access point, reporting the location of user attached monitor device 410 may be done via the public Wi-Fi access point in place of the cellular communication link. In another case where user attached monitor device 410 is operating in a low battery mode, reporting the location of user attached monitor device 410 may be done via user detached monitoring device 420 coupled using local communication link 459.

Which technologies are used to update the location of user attached monitor device 410 may be selected either by default, by programming from a central monitor system (not shown), or based upon scenarios. For example, it may be determined whether sufficient battery power as reported by power status 496 remains in user attached monitor device 410 to support a particular position determination technology. Where insufficient power remains, the particular technology is disabled. In some cases, a maximum cost of resolving location may be set for user attached monitor device 410. For example, resolving Wi-Fi location data may incur a per transaction cost to have a third party service provider resolve the location information. When a maximum number of resolution requests have been issued, the Wi-Fi position determination technology may be disabled. Further, it may be determined the likelihood that a particular position determination technology will be capable of providing meaningful location information. For example, where user attached monitor device 410 is moved indoors, GPS receiver 462 may be disabled to save power. Alternatively, where the tracking device is traveling at relatively high speeds, the Wi-Fi receiver 488 may be disabled. As yet another example, where cellular phone jamming is occurring, support for cell tower triangulation position determination may be disabled. As yet another example, where GPS jamming is occurring, GPS receiver 462 may be disabled. As yet another example, where user attached monitor device 410 is stationary, the lowest cost (from both a monetary and power standpoint) tracking may be enabled while all other technologies are disabled. Which position determination technologies are used may be based upon which zone a tracking device is located. Some zones may be rich in Wi-Fi access points and in such zones Wi-Fi technology may be used. Otherwise, another technology such as cell tower triangulation or GPS may be used.

Controller circuit 467 of user attached monitor device 410 at times functions in conjunction with cellular transceiver 468 to send and receive data and signals through wide area communication network 450. This link at times is useful for passing information and/or control signals between a central monitoring system (not shown) and user attached monitor device 410. The information transmitted may include, but is not limited to, location information, alcohol information, and information about the status of user attached monitor device 410. Based on the disclosure provided herein, one of ordinary skill in the art will recognize a variety of information that may be transferred via wide area communication network 450.

Various embodiments of user attached monitor device 410 include a variety of sensors capable of determining the status of user detached monitor device 420, and of the individual associated therewith. For example, a status monitor 466 may include one or more of the following subcomponents: power status sensor 496 capable of indicating a power status of user detached monitor device 420. The power status may be expressed, for example as a percentage of battery life remaining. Based upon the disclosure provided herein, one of ordinary skill in the art will recognize a variety of forms in which power status may be expressed. In addition, user attached monitor device 410 includes a set of shielding sensors 469 that are capable of determining whether user attached monitor device 410 is being shielded from receiving GPS signals and/or if GPS jamming is ongoing, a set of device health indicators 454, a tamper sensor 431 capable of determining whether unauthorized access to user attached monitor device 410 has occurred or whether user attached monitor device 410 has been removed from an associated individual being monitored, a motion/proximity sensor 452 capable of determining whether user attached monitor device 410 is moving and/or whether it is within proximity of an individual associated with user detached monitor device 420, and/or an alcohol sensor 453. Such an alcohol sensor may be any alcohol sensor capable of estimating an amount of alcohol in the individual being monitored. Based upon the disclosure provided herein, one of ordinary skill in the art will recognize a variety of alcohol sensors and corresponding alcohol sensing circuitry that may be used in relation to different embodiments. Based on the disclosure provided herein, one of ordinary skill in the art will recognize a variety of shielding sensors, a variety of device health transducers and indicators, a variety of tamper sensors, various different types of motion sensors, different proximity to human sensors, and various human body physical measurement sensors or transducers that may be incorporated into user attached monitor device 410 according to various different instances and/or embodiments.

Turning to FIG. 4d, a user attached monitor device 4089 is shown with an example attachment element 4090 connected at opposite ends of user attached monitor device 4089 (i.e., a first end 4097 and a second end 4098). Attachment element 4090 is operable to securely attach a tracking device 4095 (i.e., a combination of user attached monitor device 4089 and attachment element 4090) to a limb of an individual in accordance with some embodiments. In various embodiments, attachment element 4090 includes electrically and/or optically conductive material used to make a conductive connection form first end 4097 to second end 4098 through attachment element 4090 and is used in relation to determining whether user attached monitor device 4089 remains attached and/or has been tampered with. While FIG. 4d shows a strap as an example attachment element, based upon the disclosure provided herein, one of ordinary skill in the art will recognize other types of attachment elements that may be used in relation to different embodiments.

Turning to FIG. 5, a flow diagram 500 shows a method in accordance with some embodiments for maintaining a count of remaining doses of a medication and to automatically order refills of the medication. At the outset the number of remaining doses is initialized as zero. Following flow diagram 500, it is determined whether the monitored individual received a refill of their medication (bock 502). This may be determined, for example, by a response from a refilling pharmacy indicating that a prescription has been picked up by the monitored individual. Where a refill is received (block 502), the number of doses in the refill is added to the remaining doses (block 504) and the number of remaining doses is stored to memory (block 506).

Alternatively, where a refill is not received (block 502), it is determined whether a does of medication has been taken (block 508). Such a determination may be made, for example, via a message received from block 262 discussed above in relation to FIG. 2c. Where a dose of medication has been taken (block 508), one dose is subtracted from the remaining doses (block 510) and the number of remaining doses is stored to memory (block 512).

It is determined whether the number of remaining doses is less than a threshold value (block 514). In some cases, the threshold value is selected to be one week worth of doses. Based upon the disclosure provided herein, one of ordinary skill in the art will recognize a variety of threshold values that may be used in relation to different embodiments. Where the number of remaining doses is less than the threshold value (block 514), a refill of the medication is automatically ordered (block 516). This may be done, for example, by sending an electronic message to a pharmacy local to the monitored individual requesting a refill of the prescription. In addition, a message is sent to the monitored individual indicating that the refill has been ordered and providing instructions on where to pick up the refill (block 518).

In conclusion, the present invention provides for novel systems, devices, and methods for monitoring individuals and/or assets. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A system for monitoring client medication, the system comprising:
a client device, the client device including:
a camera;
an electronic interface operable to communicate instructions to the client;
a wireless transceiver operable to transfer data from the client device to a remote monitoring receiver;
a computer readable medium including non-transient instructions executable by a processor to:
communicate a first instruction via the electronic interface requesting that the client identify a medication to be taken;
determine that the client has properly identified the medication to be taken;
communicate a second instruction via the electronic interface requesting that the client position the client device to capture at least one image;
receive the at least one image from the camera;
determine that the image complies with the instruction; and based upon the determination that the image complies with the instruction, transmit the at least one image via the wireless transceiver to a remote monitor device.

2. The system of claim 1, wherein the client device is a user detached monitor device.

3. The system of claim 1, wherein the client device is a combination of a user detached monitor device and a user attached monitor device.

4. The system of claim 1, wherein the computer readable medium further includes instructions executable by the processor to:
receive an indication of the medication via the wireless transceiver;
receive a schedule indicating when the medication is to be taken; and
wherein the first instruction is communicated via the electronic interface at a time identified in the schedule.

5. The system of claim 1, wherein the first instruction requests that the client position the client device such that the camera can take an image of the medication, and wherein the computer readable medium further includes instructions executable by the processor to:
transmit the image of the medication via the wireless transceiver to the remote monitor device.

6. The system of claim 1, wherein the first instruction requests that the client position the client device such that the camera can take an image of the medication, and wherein the computer readable medium further includes instructions executable by the processor to:
transmit the image of the medication via the wireless transceiver to the remote monitor device.

7. The system of claim 1, wherein the first instruction requests that the client position the client device such that the camera can take an image of the medication, and wherein the computer readable medium further includes instructions executable by the processor to:
automatically determine that the image of the medication is the medication to be taken.

8. The system of claim 1, wherein the second instruction requests that the client position the client device to capture a first image of the client's open mouth with the medication to be taken placed therein, and to subsequently capture a second image of the client's open mouth after the client has swallowed the medication to be taken.

9. The system of claim 1, wherein the second instruction requests that the client position the client device to capture a first image of the client's open mouth with the medication to be taken placed therein, and to subsequently capture a second image of the client's open mouth after the client has swallowed the medication to be taken, wherein the second image shows a top side of the client's tongue.

10. The system of claim 1, wherein the second instruction requests that the client position the client device to capture a first image of the client's open mouth with the medication to be taken placed therein, and to subsequently capture a second image of the client's open mouth after the client has swallowed the medication to be taken, wherein the second image shows a bottom side of the client's tongue.

11. The system of claim 1, wherein the electronic interface includes an audio interface.

12. The system of claim 1, wherein the electronic interface includes a visual display interface.

13. The system of claim 1, wherein determining that the image complies with the instruction includes comparing the at least one image with a previously stored image.

14. The system of claim 1, determining that the client has properly identified the medication to be taken includes determining that the client has responded to the first instruction within a preset time period.

15. The system of claim 1, determining that the image complies with the instruction includes determining that the client has responded to the second instruction within a preset time period.

16. A method for monitoring client medication, the method comprising:
sending a message to a monitored individual via a client device prompting the monitored individual to take a medication;
receiving a first image from a client device showing a container of the medication;
prompting the individual to perform a multi-step process to take the medication;
receiving a second image from the client device showing the medication inside the mouth of a monitored individual associated with the client device;
receiving a third image from the client device showing the mouth of the monitored individual with a tongue of the individual in a down position;
receiving a fourth image from the client device showing the mouth of the monitored individual with a tongue of the individual in an up position; and
based upon a combination of the first image, the second image, the third image, and the fourth image, determining that the monitored individual successfully completed taking the medication.

17. The method of claim 16, wherein the client device is a user detached monitor device.

18. The method of claim 16, wherein the client device is a combination of a user detached monitor device and a user attached monitor device.

19. The method of claim 16, wherein the determining that the monitored individual successfully completed taking the medication is done automatically.

20. A system for monitoring client medication, the system comprising:
a client device and a central monitoring station;
wherein the client device includes:
a camera;
an electronic interface operable to communicate instructions to the client;
a wireless transceiver operable to transfer data from the client device to a remote monitoring receiver;
a first computer readable medium including non-transient instructions executable by a processor to:
communicate a first instruction via the electronic interface requesting that the client identify a medication to be taken;
determine that the client has properly identified the medication to be taken;
communicate a second instruction via the electronic interface requesting that the client position the client device to capture at least one image;
receive the at least one image from the camera;
determine that the image complies with the instruction; and based upon the determination that the image complies with the instruction, transmit the at least one image via the wireless transceiver to a remote monitor device; and
wherein the central monitoring station includes:
a second computer readable medium including non-transient instructions executable by a processor to:
receive the at least one image.
